(19) United States
(12) Patent Application Publication
(10) Pub. No.: US 2024/0272101 A1
(43) Pub. Date: Aug. 15, 2024

(54) Direct Sampling from Phase-Separated Sample for Hydrophobicity Assessment

(71) Applicant: DH Technologies Development Pte. Ltd., Singapore (SG)

(72) Inventors: Thomas COVEY, Concord (CA); Chang LIU, Richmond Hill (CA); Yuzhu GUO, Kleinburg (CA)

(21) Appl. No.: 18/568,410

(22) PCT Filed: Jun. 8, 2022
(86) PCT No.: PCT/IB2022/055353

§ 371 Date: Dec. 8, 2023

Related U.S. Application Data
(60) Provisional application No. 63/208,699, filed on Jun. 9, 2021.

(51) Int. Cl. G01N 23/2208; G16C 20/30

(52) U.S. Cl. CPC G01N 23/2208; G16C 20/30; G01N 2223/071

(57) ABSTRACT

In one aspect, a method of assessing hydrophobicity of a target analyte is disclosed, which includes introducing a sin-gle-phase system containing a concentration of the target analyte into a mass spectrometer to acquire at least one mass signal associated with the target analyte, introducing a phase-separated system (e.g., a two-phase system) containing substantially the same concentration of the target analyte into the mass spectrometer to acquire at least one mass signal associated with said target analyte, and utilizing a ratio of the intensities of the mass signals to assess hydrophobicity of the target analyte.

### BACKGROUND

The present teachings are generally directed to mass spectrometric methods and systems for assessing hydrophobicity of a target analyte.

The hydrophobicity of a target analyte is an important property that can be used to assess the feasibility of oral administration of a target analyte, e.g., when used as an active ingredient of a therapeutic formulation. In particular, the pharmacokinetics of an analyte in the human body including its absorption, distribution, metabolism, and extraction depends, at least in part, on the hydrophobicity of the analyte. A parameter known as a partition coefficient is typically employed to assess the oral bioavailability of an analyte. A partition coefficient P can defined as the ratio of concentration of an analyte in a mixture of two immiscible solvents at equilibrium and its measured value may be employed to assess the oral bioavailability of the analyte.

### SUMMARY

In one aspect, a method for assessing hydrophobicity of a target analyte (which is herein also referred to as a target compound) is disclosed, which includes introducing a single-phase system containing a quantity of the target analyte into a mass spectrometer to acquire a first measurement of at least one mass signal associated with the target analyte, introducing at least one phase of a phase-separated system (e.g., a two-phase system), which contains substantially the same quantity of the analyte into the mass spectrometer to acquire a second measurement of at least one mass signal associated with the analyte, and utilizing the first and the second measurements to assess hydrophobicity of the target analyte.

The mass measurement of a phase (e.g., an organic phase) of the phase-separated system is typically performed after the system has reached equilibrium. For example, sufficient time is allowed for the concentration of a target analyte within an aqueous phase and an organic phase of a two-phase system to reach their equilibrium concentrations.

In some embodiments in which the volume of the aqueous phase is different than the volume of the organic phase, a volume scaling factor can be utilized for obtaining an accurate measure of the hydrophobicity of the target analyte.

Although in some embodiments the quantity of the target analyte in the single-phase and the phase-separated system is known, in general, various embodiments of the present teachings only require that a quantity of the target analyte (e.g., the number of moles of the target analyte) in the single-phase and the two-phase systems be substantially the same, even if the absolute quantity of the target analyte may not be known. In other words, in embodiments of the present teachings, the relative quantities of the target analyte in the single-phase and two-phase system should be substantially equal to unity.

The single-phase system and the two-phase system can be introduced into the mass spectrometer using a variety of different techniques. By way of example, and without limitation, the single-phase system and the two-phase system can be introduced into the mass spectrometer via application of one or more acoustic pulses thereto so as to eject a plurality of droplets into the mass spectrometer. For example, the acoustic pulse(s) can be applied to an exposed surface of the single-phase system and/or that of the two-phase system to eject the droplets into the mass spectrometer.

In some embodiments, the single-phase system can include an aqueous solution of the target analyte and the two-phase system can include a mixture of the target analyte with water and an organic solvent. By way of example, in some embodiments, the two-phase system can be prepared by adding an organic solvent to an aqueous solution of the target analyte. A variety of organic solvents can be employed. Some examples of suitable organic solvents include, without limitation, pentanol and octanol.

In some embodiments, the single phase system can be in the form of a single organic phase (e.g., one prepared via mixing a target analyte with one or more organic solvent(s)). In some such embodiments, the two-phase system can be prepared by adding an aqueous solution to the organic phase. Further, in some embodiments, the measurements of a mass signal of the target analyte using the single-phase system and the two-phase system can be acquired via introduction of one or more samples of at least a portion of the single-phase system and the two-phase system into a mass spectrometer. As discussed in more detail below, these measurements of the mass signal associated with the target analyte can be used to assess the hydrophobicity of the target analyte.

As noted above, in some embodiments, a mass signal of the target analyte acquired using the two-phase system can be compared with a respective mass signal of the target analyte acquired using the single-phase system to assess the hydrophobicity of the target analyte. For example, a ratio of the two measurements of the mass signals may be used to assess the hydrophobicity of the target analyte. For example, when the measurement of the mass signal from the two-phase system is achieved via sampling an organic phase of the two-phase system (e.g., via sampling from the top organic layer if the organic phase has a lower density than the aqueous phase) and the single-phase system is an aqueous system, an increase in the ratio of the measurements of the mass signal from the two-phase system relative to the single-phase system indicates an increase in the hydrophobicity of the target analyte. Alternatively, an increase in the intensity of a mass signal associated with an aqueous phase relative to the respective mass signal associated with an organic phase indicates a decrease in the hydrophobicity of the target analyte.

Without being limited to any particular theory, an increase in the ratio of the measurements of a mass signal from an organic phase relative to an aqueous phase indicates a higher degree of the solubility of the target compound in the organic phase, thereby indicating a greater hydrophobicity of the target compound.

In some embodiments, a ratio of a mass signal associated with an organic phase of the two-phase system and a respective mass signal associated with an aqueous phase of a single phase system can be utilized to assess the oral bioavailability of the target analyte as a therapeutic agent. By way of example, in some embodiments, when such a mass ratio lies within a range of about −0.4 to about +5.6, the target analyte is considered as exhibiting an acceptable oral bioavailability. Conversely, when a ratio of a mass signal associated with an aqueous phase relative to the respective mass signal associated with an organic phase is employed for assessing the hydrophobicity of a target analyte, as the ratio increases the hydrophobicity of the target analyte decreases.

In a related aspect, a method of assessing hydrophobicity of a target analyte is disclosed, which includes acquiring a first measurement of a mass signal of a target analyte from a single-phase system containing a quantity of said target analyte, acquiring a second measurement of the respective mass signal of the target analyte from a two-phase system containing substantially the same quantity of said target analyte, and comparing the second and the first measurements of the mass signal to assess hydrophobicity of said target analyte.

In some embodiments, the hydrophobicity of the target analyte can be assessed by computing a ratio of an intensity of the second measurement of the mass signal relative to that of the first measurement of the mass signal. In some such embodiments, the computed ratio can be used to calculate a partition coefficient (P) for the target analyte based on that ratio. In some embodiments, the logarithm of the partition coefficient (log(P)), which is herein referred to as the “hydrophobicity parameter,” can be used for assessing the hydrophobicity and the oral bioavailability of the target analyte. By way of example, in some embodiments in which the hydrophobicity parameter is calculated based on the ratio of the measurement of the mass signal from an aqueous phase of the two-phase system relative to the respective measurement from an aqueous single-phase system, when the hydrophobicity parameter is within a range of about −0.4 to about +5.6, the target analyte is considered as having an acceptable oral bioavailability.

In a related aspect, a system for determining hydrophobicity of a target analyte is disclosed, which includes a single-phase specimen containing a quantity of the target analyte, a two-phase specimen containing substantially the same quantity of the target analyte, and a mass spectrometer that is configured to acquire at least one mass signal of the target analyte. The mass spectrometer can be utilized to acquire two measurements of a mass signal of the target analyte, where one of the measurements is associated with the single-phase specimen and the other measurement is associated with the two-phase specimen. An analysis module can receive the two measurements of the mass signal and utilize those measurements to assess the hydrophobicity (and hence oral bioavailability) of the target analyte.

For example, the analysis module can be configured to compute a ratio of the intensities of the mass signals (e.g., a ratio of the intensity of the mass signal measurement associated with the two-phase system relative to the intensity of the mass signal measurement associated with the single-phase system). The analysis module can further employ the computed ratio to calculate a hydrophobicity parameter associated with the target analyte, e.g., as a logarithm of the partition coefficient.

Moreover, the analysis module can be configured to determine whether the computed hydrophobicity parameter lies within a predefined range for assessing the hydrophobicity (and hence the oral bioavailability) of the target analyte. By way of example, the predefined range may extend from about −0.4 to about +5.6. By way of example, when the ratio is indicative of the intensity of the mass signal associated with an organic phase of the two-phase system relative to the intensity of the mass signal associated with an aqueous single-phase system, the analysis module can be configured to indicate that the target analyte exhibits a satisfactory oral bioavailability when the computed hydrophobicity of the target analyte, calculated based on that ratio (e.g., as a logarithm of the partition coefficient) lies within this range, i.e., when the hydrophobicity parameter is in the range of about −0.4 to about +5.6.

In some embodiments, the mass spectrometer can include an open port interface (OPI) for receiving a sample of any of the single-phase and the two-phase specimen and an acoustic actuator that can apply acoustic pulses to an exposed interface (e.g., a top surface) of the single-phase or the two-phase specimen for ejecting a plurality of droplets into the mass spectrometer. While in some embodiments the acoustic actuator can be coupled to a sample holder in which the single-phase and the two-phase systems are contained so as to cause the ejection of a plurality of droplets from a top surface of the specimen, in other embodiments, the sample holder and the acoustic actuator can be configured such that the acoustic actuator can be used to eject droplets of the single-phase and the two-phase specimens via application of acoustic pulses to the bottom surface of the respective specimen.

In other embodiments, the mass spectrometer can employ other mechanisms for introducing samples of the single-phase and the two-phase specimens into the mass spectrometer. By way of example, a sample ejection system marketed by CellLink Life Sciences of Phoenix, U.S.A. under the trade designation i-Dot (Immediate Drop-on-Demand Technology) employs a plurality of positive controlled pressure pulses to generate droplets from about 8 to about 50 nanoliters from a small opening at the bottom of a sample holder, e.g., a well in which the single-phase or the two-phase system is contained. In another embodiment, a micro-dispensing technology marketed by PolyPico Technologies, Ltd. of Ireland can be employed for ejecting droplets from the single-phase system and/or a phase of a two-phase system.

Further understanding of various aspects of the present teachings can be obtained by reference to the following detailed description

### BRIEF DESCRIPTION OF THE DRAWINGS

FIG. 1 is a flow chart depicting various steps of a method according to an embodiment of the present teachings for assessing hydrophobicity of a target analyte,

FIGS. 2A and 2B schematically depict a single-phase specimen and a two-phase specimen, which can be employed in embodiments of the present teachings to assess hydrophobicity of a target analyte contained in those specimens,

FIG. 3 schematically depicts a mass spectrometric system according to an embodiment of the present teachings for assessing the hydrophobicity of a target analyte,

FIG. 4 schematically depicts a two-phase system contained in a sample holder and an acoustic actuator operably coupled to the sample holder for applying one or more acoustic pulses to one layer of the two-phase system for ejecting portions of that layer as a plurality of droplets into an open port interface of a mass spectrometer,

FIG. 5 shows the reflection of an acoustic wave applied to the two-phase system of FIG. 4 from different interfaces of the sample holder and the two-phase system,

FIG. 6 presents data corresponding to a plurality of measurements of a mass signal associated with a target analyte, where the mass measurements are acquired using an acoustic actuator to eject droplets of a two-phase system containing the target analyte into a mass spectrometer,

FIG. 7A provides a plurality of measurements of a mass signal for dextromethorphan acquired from a water/pentanol two-phase system containing dextromethorphan,

FIG. 7B provides a plurality of measurements of a mass signal for adenosine acquired from a water/pentanol two-phase system containing adenosine.

### DETAILED DESCRIPTION

It will be appreciated that for clarity, the following discussion will explicate various aspects of embodiments of the present disclosure, while omitting certain specific details wherever convenient or appropriate to do so. For example, discussion of like or analogous features in alternative embodiments may be somewhat abbreviated. Well-known ideas or concepts may also for brevity not be discussed an any great detail. One of ordinary skill will recognize that some embodiments of the present disclosure may not require certain of the specifically described details in every implementation, which are set forth herein only to provide a thorough understanding of the embodiments. Similarly it will be apparent that the described embodiments may be susceptible to alteration or variation according to common general knowledge without departing from the scope of the disclosure. The following detailed description of embodiments is not to be regarded as limiting the scope of the applicant's teachings in any manner.

As used herein, the terms “about” and “substantially equal” refer to variations in a numerical quantity that can occur, for example, through measuring or handling procedures in the real world; through inadvertent error in these procedures; through differences in the manufacture, source, or purity of compositions or reagents; and the like. Typically, the terms “about” and “substantially” as used herein means 10% greater or lesser than the value or range of values stated or the complete condition or state. For instance, a concentration value of about 30% or substantially equal to 30% can mean a concentration between 27% and 33%. The terms also refer to variations that would be recognized by one skilled in the art as being equivalent so long as such variations do not encompass known values practiced by the prior art.

As used herein and conventionally understood, two substances are “miscible” when they can be mixed in all proportions to form a homogeneous mixture. For example, water and alcohol are miscible because they can be mixed in all proportions to form a homogenous mixture. Further, two substances are immiscible when their mixture does not result in a homogeneous mixture.

The present teachings are generally directed to high-throughput mass spectrometric (MS) systems and methods for assessing the hydrophobicity of a target analyte based on the comparison of the MS signal of the target analyte from MS analysis of samples from a single-phase system and a phase-separated system.

For example, a single-phase system and a two-phase system having substantially the same quantity of a target analyte can be prepared and mass signals of the target analyte using the single-phase system and the two-phase system can be acquired. The mass signals can be compared to assess the hydrophobicity of the target analyte. For example, a ratio of an intensity of a mass signal associated with an organic phase of the two-phase system relative to an intensity of a respective mass signal associated with an aqueous single-phase system can be positively correlated with the hydrophobicity of the target analyte, i.e., this ratio increases as the hydrophobicity of the target analyte increases. In another embodiment, the ratio of an intensity of a mass signal associated with an aqueous phase of the two-phase system relative to an intensity of a respective mass signal associated with an organic phase of a single-phase system can be negatively correlated with hydrophobicity of the target analyte, i.e., this ratio decreases as the hydrophobicity of the target analyte increases. Thus, the ratio of the intensity of the respective mass signals can be employed as a measure of hydrophobicity of the target analyte.

In conventional methods in which mass spectrometry is employed for assessing the hydrophobicity of a target analyte, a two-phase system containing the analyte is prepared and each phase is isolated from the two-phase system and injected into the mass spectrometer system for analysis.

In comparison with such conventional methods, embodiments of the mass spectrometric methods and systems according to the present teachings can provide a higher throughput and require less sample preparation.

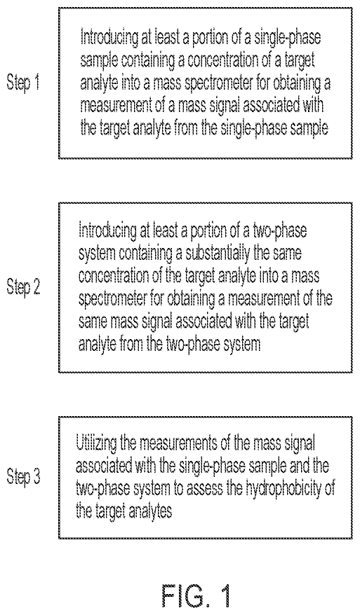

With reference to the flow chart of FIG. 1, in one embodiment of a method according to the present teachings for assessing hydrophobicity of a target analyte, a single-phase system containing a quantity of the target analyte is introduced into a mass spectrometer and a measurement (e.g., an intensity measurement) of at least one mass signal associated with the target analyte is acquired (step 1). A phase-separated system (e.g., a two-phase system) containing substantially the same quantity of the target analyte is prepared, and a phase of the phase-separated system in which the miscibility of the target analyte is different that its miscibility in the single phase system is introduced into the mass spectrometer (or a different mass spectrometer) and a respective measurement of said at least one mass signal associated with the target analyte is acquired (step 2). The measurements of the mass signals are utilized to assess the hydrophobicity of the target analyte (step 3).

For example, in some embodiments, the comparison of the measurements of the mass signals can be performed by obtaining a ratio of an intensity of the mass signal of the target analyte acquired using the phase-separated system (e.g., the two-phase system) relative to an intensity of a respective mass signal of the target analyte acquired using the single-phase system.

The acquisition of the mass signal of the target analyte using the single-phase system and the two-phase system can be performed in any order, or concurrently (e.g., using two different mass spectrometers). Although the acquisition of the mass signals associated with the target analyte can be achieved using different mass spectrometers, typically, the same calibrated mass spectrometer is employed for acquisition of the two measurements of the mass signal to ensure that the observed relative intensities of the mass signals can be used to obtain an accurate assessment of the hydrophobicity of the target analyte. Further, as noted above, in some embodiments in which the volume of the aqueous and the organic phases are different, a volume scaling factor can be utilized to normalize the mass signal measurements.

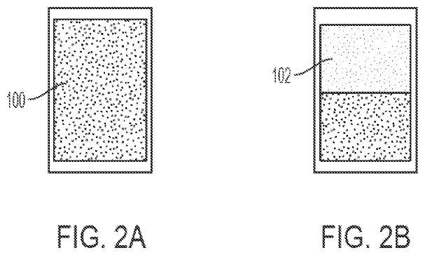

With reference to FIGS. 2A and 2B, by way of example, in some embodiments, two specimens 100 and 102 can be prepared, where the specimen 100 is a single-phase system and the specimen 102 is a two-phase system. By way of example, a quantity of a target analyte (herein also referred to as a target compound) can be dissolved in water to form a standard single-phase system, e.g., a standard aqueous solution, and an organic solvent can be added to the standard single-phase system to form a two-phase system. A variety of different organic solvents can be employed. Some examples of suitable organic solvents include, without limitation, pentanol and octanol.

As noted above, in some embodiments, a mass signal associated with the target analyte can be acquired by introducing at least a portion of the single-phase system into a mass spectrometer and obtaining a mass signal thereof. Further, another mass signal associated with the target analyte can be acquired by introducing at least a portion of the two-phase system (e.g., the phase of the two-phase system in which the miscibility of the target analyte is different than that in the single-phase system) into the mass spectrometer and obtaining a respective mass signal of the target analyte.

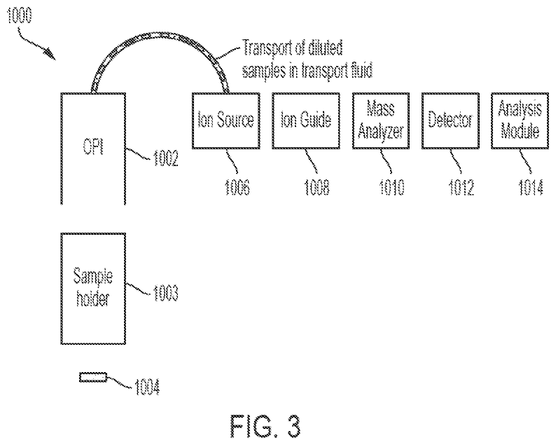

As shown schematically in FIG. 3, in some embodiments, a mass spectrometer 1000 having an open port interface (OPI) 1002 can be employed for acquiring the mass signals of the target analyte associated with the single-phase system as well as the two-phase system. In some such embodiments, an acoustic droplet dispenser 1004 (herein also referred to as an acoustic actuator) can be operably coupled to a sample holder 1003 in which the single-phase system and the two-phase system are disposed so as to eject a plurality of droplets thereof into the OPI interface of the mass spectrometer.

In this embodiment, the ejection of the droplets from the single-phase system is achieved using an acoustic droplet dispenser equipped with a single-phase calibration system and the ejection of the droplets from the two-phase system is achieved using a two-phase acoustic calibration system.

In embodiments in which the absolute value of log(P) is required, such calibration of the acoustic droplet dispenser can ensure that the ejection of the droplets from the single-phase system and the two-phase system will result in the introduction of substantially the same amount of the single-phase system and the two-phase system into the mass spectrometer. This can, in turn, ensure that an observed intensity difference associated with two measurements of a mass signal of the target analyte, where one measurement is acquired using the single-phase system and the other the two-phase system, can be accurately attributed to the hydrophobicity of the target analyte, rather than potential differences in the amounts of the single-phase system and the two-phase system introduced into the mass spectrometer.

In some embodiments, rather than the absolute value of log(P), the relative hydrophobicity of a plurality of target analytes is needed, the calibration of the volumes of the single-phase and the phase-separated systems ejected into a mass spectrometer may not be critical as long as the preparation of the different samples and the way the samples are analyzed remain substantially the same among those sample (for example, substantially the same volumes of the aqueous and the organic phases as well as the quantity of the target analyte are employed).

In this embodiment, the OPI interface 1002 carries the introduced sample including the target analyte to an atmospheric pressure ionization source (API) source, which ionizes at least a portion of the target analyte so as to generate a plurality of ions. The ions are received by an ion guide that focuses the ions to generate an ion beam, which is received by at least one downstream mass analyzer (e.g., one or more quadrupole and/or time-of-flight mass analyzers).

At least a portion of the ions passing through the mass analyzer is received by a downstream ion detector 1012, which generates ion detection signals in response to the detection of the ions. An analysis module 1014 can receive the ion detection signals generated by the ion detector and can generate information regarding mass-to-charge ratio (m/z) of the target analyte and/or fragments thereof.

In some embodiments, two measurements of at least one MRM (multiple reaction monitoring) transition of the target analyte, acquired via MS/MS analysis of at least a portion of the single-phase system and at least a portion of the two-phase system, can be utilized as mass signals for assessing the hydrophobicity of the target analyte.

By way of example, the analysis module can determine a ratio of an intensity of a mass signal obtained using the two-phase system relative to an intensity of a respective mass signal obtained using the single-phase system to compute a measure of the hydrophobicity of the target analyte. In embodiments in which the ratio corresponds to the intensity of the mass signal acquired from an organic phase of the two-phase system relative to the intensity of the mass signal acquired from an aqueous single-phase system, due to a higher efficiency associated with the extraction of a hydrophobic target analyte from an organic matrix, the ratio increases as the hydrophobicity of the target analyte increases.

A hydrophobicity parameter indicative of hydrophobicity of a target analyte can be computed as follows:

$$Log ( P ) = \frac{\begin{matrix}Concentration of target analyte \\ in an organic phase ( e . g . , Ocotanol )\end{matrix}}{\begin{matrix}concentration of target analyte \\ in an aqueous phase ( e . g . , water )\end{matrix}} \qquad Eq . ( 1 )$$

By way of example, in some embodiments in which the volume of the organic (or the aqueous phase) in the two-phase system is substantially the same as the volume of the aqueous (or organic phase) in the single-phase system (e.g., a single-phase system formed of 25 μL of water and a two-phase system formed of 25 μL of Octanol and 25 μL of water), the hydrophobicity parameter (Log(P)) may be calculated as follows, assuming that substantially the same droplet sizes of the two phases are introduced into the mass spectrometer and substantially no ionization suppression:

$$Log ( P ) = \frac{A}{B} , \qquad Eq . ( 2 )$$

where,
   A=intensity of mass signal from an organic phase of the two-phase system, and
   B=intensity of a respective mass signal from an aqueous single phase system−(minus) intensity of mass signal from an organic phase of the two-phase system.

If the volumes of the single-phase system and a phase of the two-phase system introduced into the mass spectrometer are different, then a volume factor can be added to the above Eq. (2) to correct such a different in the volumes. If the intensity resulting from the ejection of the same amount of the analyte out of the aqueous and organic phase are different (e.g. due to the different levels of the ionization suppression), additional correction factors could be added in to the above Eq. (2).

In some embodiments, the analysis module can compare the hydrophobicity parameter with a predefined range to assess the oral bioavailability of the target analyte. For example, the analysis module can compare the hydrophobic parameter computed via the above Equation (2) with a range of about −0.4 to about +5.6 and if the hydrophobic parameter is within this range, the analysis module can indicate that the target analyte is a good candidate for use as a therapeutic agent for oral administration, e.g., when utilized as an active ingredient of a pharmaceutical formulation.

In embodiments in which an acoustic droplet dispenser is used to introduce the target analyte into an open interface port of a mass spectrometer, a high throughput workflow can be achieved due to the high throughput of sample preparation and sample introduction into the mass spectrometer. By way of example, in some embodiments, the two-phase system can be automatically generated via addition of a predefined amount of an organic solvent (e.g., pentanol) to a single phase aqueous solution of the target analyte followed by mass analysis of the single phase aqueous solution and the two-phase system.

The following examples are provided for further elucidation of various aspects of the present teachings, and are not provided to indicate necessarily the optimal ways of practicing the present teachings and/or the optimal results that can be obtained. In particular, although the examples below are described in connection with the use of pentanol/water system as a two-phase system and the use of an acoustic actuator for sampling the two-phase system from the exposed surface of a top pentanol layer, the present teachings are not limited to such embodiments.

For example, in some embodiments, an octanol/water mixture may be employed to prepare the two-phase system.

Further, in some embodiments, the two-phase system can include a top aqueous layer and a bottom organic layer. By way of example, and without limitation, an example of such a two-phase system is a water/dichloromethane system in which the aqueous phase is on the top and the organic phase at the bottom.

In yet another embodiment, a sampling system capable of sampling a bottom layer of a two-phase system may be employed, e.g., using the aforementioned i-Dot system.

### EXAMPLES

### Example 1

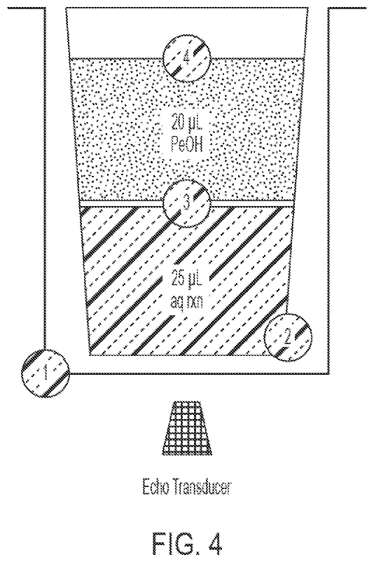

A two-phase system was prepared by adding 20 μL of pentanol to 25 μL of an aqueous solution of dextromethorphan. A SCIEX Triple Quad 6500+ mass spectrometer system having an acoustic droplet dispenser (shown schematically in FIG. 4) equipped with a two-layer acoustic calibration was employed to eject a plurality of droplets from the two-phase system into an OPI of the mass spectrometer for observing an MRM transition of the target analyte. Although in this example, a single MRM transition was monitored, in other cases, multiple MRM transitions of the target analyte can be monitored.

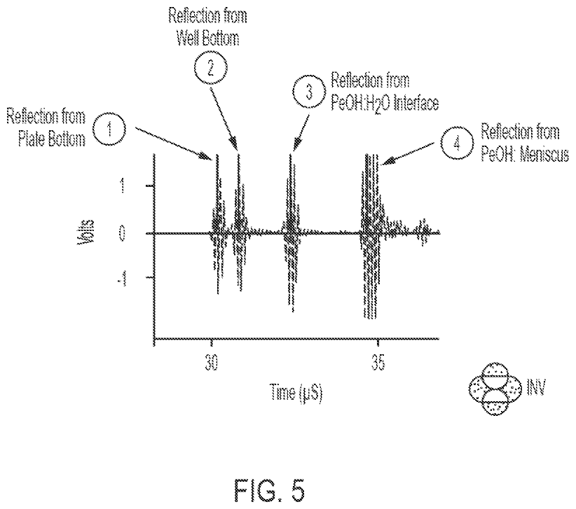

As shown in FIG. 5, in this example, the acoustic dispenser can generate and focus acoustic energy at the top surface of the two-phase system to eject droplets from the top layer of the two-phase system into the OPI of the mass spectrometer.

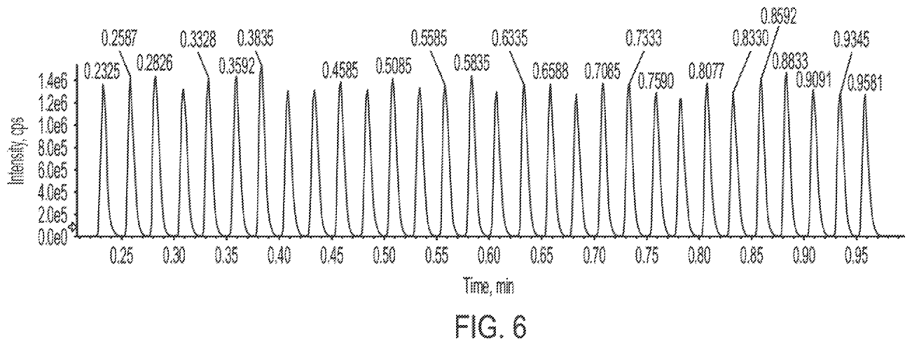

FIG. 6 presents the total ion current (TIC) corresponding to a plurality of measurements of an MRM transition of the target analyte, illustrating that the acoustic droplet dispenser can provide stable and reproducible ejection of the target analyte into the mass spectrometer.

### Example 2

The hydrophobicity of dextromethorphan and adenosine were determined using embodiments of the present teachings.

A single-phase aqueous sample was prepared as 1 μg/ml of dextromethorphan in 25 μL of water. A two-phase system was then formed by adding 25 μL of pentanol to 25 μL water in which 0.025 μg was mixed.

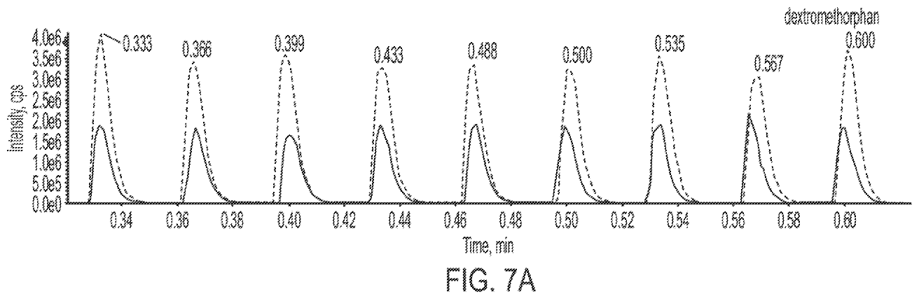

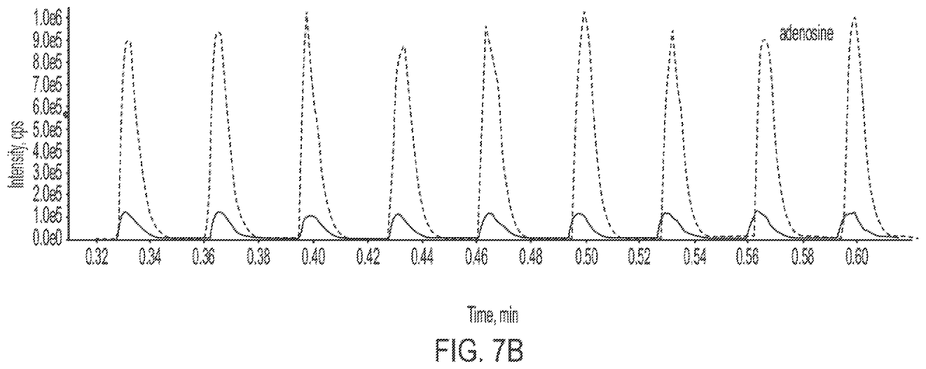

The above mass spectrometer was then utilized to obtain a series of measurements of the 272.2→215.1 MRM transition of the target analyte via sampling the single-phase solution and the two-phase system. FIG. 7A shows the mass signals of the target analyte associated with the single-phase solution and the two-phase system.

By way of comparison, a single-phase aqueous sample of adenosine was prepared by mixing adenosine with 25 μL of water to prepare a single-phase aqueous solution having a concentration of 1 μg/mL of adenosine. A two-phase system was prepared by adding 25 μL of pentanol to 25 μL aqueous single-phase system of adenosine having a concentration of 1 μg/mL adenosine.

The above mass spectrometer was then utilized to obtain a series of measurements of the 268.1→136.1 MRM transition of the target analyte via sampling the single-phase solution and the two-phase system. FIG. 7B shows the mass signals of the target analyte associated with the single-phase solution and the two-phase system of adenosine.

The above data is consistent with known differences in the hydrophobicity of dextromethorphan and adenosine. In particular, adenosine has a log P of −1.05 while dextromethorphan has a log P of +3.75, indicating that dextromethorphan is more hydrophobic than adenosine. The difference between the hydrophobicity of dextromethorphan and adenosine is reflected in the data presented in FIGS. 7A and 7B.

In particular, the relative ratio of the two-phase sampling signal versus the aqueous sampling signal associated with adenosine is less than that associated with dextromethorphan, thus indicating that methods and systems according to the present teachings can be used to assess the hydrophobicity of a target analyte.

Although some aspects have been described in the context of a system and/or an apparatus, it is clear that these aspects also represent a description of the corresponding method, where a block or device corresponds to a method step or a feature of a method step. Analogously, aspects described in the context of a method step also represent a description of a corresponding block or item or feature of a corresponding apparatus. Some or all of the method steps may be executed by (or using) a hardware apparatus, like for example, a processor, a microprocessor, a programmable computer or an electronic circuit. In some embodiments, some one or more of the most important method steps may be executed by such an apparatus.

Depending on certain implementation requirements, embodiments of the invention can be implemented in hardware and/or in software. The implementation can be performed using a non-transitory storage medium such as a digital storage medium, for example a floppy disc, a DVD, a Blu-Ray, a CD, a ROM, a PROM, and EPROM, an EEPROM or a FLASH memory, having electronically readable control signals stored thereon, which cooperate (or are capable of cooperating) with a programmable computer system such that the respective method is performed. Therefore, the digital storage medium may be computer readable.

It should be understood that the methods and systems according to the present teachings can be employed for predicting the cleavage products of a variety of macromolecules.

What is claimed is:

1. A method of assessing hydrophobicity of a target analyte, comprising:
preparing a single-phase system and a phase-separated system containing substantially the same quantity of a target analyte,
introducing at least a portion of the single-phase system into a mass spectrometer to acquire a first measurement of at least one mass signal associated with said target analyte,
introducing at least a portion of a phase of the phase-separated system in which miscibility of said target analyte is different than its miscibility in said single-phase system into the mass spectrometer to acquire a second measurement of said at least one mass signal associated with said target analyte, and
utilizing said first and second measurements of said mass signal to assess hydrophobicity of said target analyte.

2. The method of claim 1, wherein any of said steps of introducing any of said single-phase system and said phase-separated system into the respective mass spectrometer comprises applying one or more acoustic pulses to any of said single-phase system and said phase phase-separated system so as to eject a plurality of droplets thereof into the respective mass spectrometer.

3. The method of claim 2, wherein said one or more acoustic pulses are applied to an exposed surface of any of said single-phase and phase-separated system.

4. The method of claim 1, wherein said single-phase system comprises an aqueous solution of said target analyte.

5. The method of claim 1, wherein said phase-separated system comprises a mixture of said target with water and an organic solvent, and wherein said organic solvent optionally includes any of pentanol and octanol.

6. The method of claim 1, wherein said step of utilizing said first and said second measurements for assessing hydrophobicity of the target analyte comprises obtaining a ratio of said second and said first mass measurements and determining whether said ratio lies within a predefined range.

7. The method of claim 7, wherein said predefined range extends from about −0.4 to about 5.6.

8. The method of claim 6, wherein said ratio increases as hydrophobicity of said target analyte increases.

9. A method of assessing hydrophobicity of a target analyte, comprising:
acquiring a first measurement of a mass signal of a target analyte from a single-phase system containing a quantity of said target analyte,
acquiring a second measurement of said mass signal of the target analyte from a phase of a phase-separated system, which contains substantially the same quantity of said target analyte, wherein said target analyte exhibits a miscibility in said phase of the phase-separated system that is different than its miscibility in said single-phase system, and
utilizing said second and said first measurements of the mass signal to assess hydrophobicity of said target analyte.

10. The method of claim 9, wherein said step of utilizing said second and first measurements of the mass signal comprises computing a ratio of an intensity of said second measurement of the mass signal relative to said first measurement of the mass signal.

11. The method of claim 9, further comprising calculating a hydrophobicity parameter for said target analyte based on said ratio.

12. The method of claim 11, further comprising computing said hydrophobicity parameter associated with said target analyte as a logarithm of said partition coefficient (log(P)).

13. The method of claim 11, further comprising determining whether said hydrophobicity parameter lies within a predefined range for assessing hydrophobicity of said target analyte.

14. The method of claim 14, wherein said predefined range extends from about −0.4 to about +5.6.

15. A system for determining hydrophobicity of a target analyte, comprising:
a single-phase system containing a quantity of said target analyte,
a phase-separated system containing substantially the same quantity of said target analyte,
a mass spectrometric system configured to acquire a measurement of at least one mass signal of said target analyte, and
an analysis module configured to receive a plurality of measurements of the mass signal associated with said single-phase and said phase-separated system and utilize said measurements to compute a measure of hydrophobicity of said target analyte.

16. The system of claim 15, wherein said analysis module is configured to compute said measure of hydrophobicity based on a partition coefficient of said target analyte.

17. The system of claim 17, wherein said analysis module is configured to compute said measure of hydrophobicity as a logarithm of said partition coefficient of said target analyte.

18. The system of claim 15, wherein said analysis module is configured to assess an oral bioavailability of said target analyte based on said computed measure of hydrophobicity.

19. The system of claim 18, wherein said analysis module is configured to indicate that said target analyte exhibits an acceptable oral bioavailability when said logarithm of the partition coefficient is in a range of about −0.4 to about 5.6.
